(19) United States
(12) Patent Application Publication
(10) Pub. No.: US 2024/0115207 A1
(43) Pub. Date: Apr. 11, 2024

(54) SYSTEM AND METHOD FOR SENSING VITAL SIGNS OF HUMAN AND ANIMAL USING AN ARRAY OF 3D-MICRO ELECTRO-MECHANICAL SYSTEM (MEMS) SENSORS FOR REMOTE HEALTH MONITORING

(71) Applicant: OnSky Health International, San Jose, CA (US)

(72) Inventor: Hung Cao Nguyen, San Jose, CA (US)

(21) Appl. No.: 17/961,105

(22) Filed: Oct. 6, 2022

(51) Int. Cl. A61B 5/00; A61B 5/0205

(52) U.S. Cl. CPC A61B 5/6892; A61B 5/0205; A61B 5/7225; A61B 5/0816

(57) ABSTRACT

A system to sense human and animal vital signs using an array of three-dimensional sensor units for remote health monitoring, including: three-dimensional sensor units each configured to sense heartbeats and respiratory pulses along x, y and z axes from any location on a surface of a mattress or other medium in which a human or animal is on, wherein each sensor outputs three analog signals representing the three dimensions being sensed; three analog-to-digital converters (ADC) corresponding to each of the three-dimensional sensor units to convert the three output analog signals to three digital signals; a micro-controller unit (MCU) configured to perform digital signal processing (DSP) on the digital signals for each ADC and to output the digitized signals; and a finite impulse response (FIR) low-pass filter (LPF) to low-pass filter each of the output digitized signals.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C.F.R 1.71(d).

### BACKGROUND OF THE INVENTIVE CONCEPT

### 1. Field of the Invention

The present inventive concept relates to a system and method for sensing human vital signs using an array of 3D-MEMS sensors for remote health monitoring. More particularly, but not exclusively, this inventive concept relates to a system and method for sensing humans' and animals' vital signs using an array of 3D-MEMS sensors for remote health monitoring wherein optimum weights can be calculated for each sensor output to obtain the optimum signal of heartbeats and respiration pulses.

### 2. Description of the Related Art

Humans spend approximately one third of their life sleeping as sleep is a necessary process for our health and longevity. However, about one in eight people die each year due to various reasons while sleeping, including issues such as obstructive sleep apnea, heart attacks, strokes, SIDS (babies), as well as other chronic diseases. Thus, while sleep is an essential process to recharge our bodies and mind, humans can be vulnerable during sleep.

A patient who is transferred from an intensive care unit (ICU) to either a recovery room or even back to their home and possibly placed under homecare is often under some form or forms of medication, including sleep medication, prescribed by a doctor as part of a recovery procedure. Unfortunately, if the patient has sleep apnea, they may not be able to wake up in time to catch their breath, especially if they are under the influence of a medication which disables their brain's ability to recognize that they need oxygen. Further, people with heart disease, hypertension or other chronic diseases may die in their sleep due to their heart suddenly stopping from beating during the middle of the night. Similar issues apply regarding animals.

Attempts have been made to monitor heart rate while a person is sleeping. For example, there is known a contact-less mechanical sensor which is integrated into an ordinary bed. This sensor records the ballistic forces due to cardiac activity and is referred to as ballistocardiogram (BCG). However, changes in the relative position of the sensor and patient occur, which leads to a high signal variability. Limb movements, coughing, snoring, etc., cause mechanical activity which superimposes to the BCG during recording.

The article titled: “Heart Rate Estimation on a Beat-to-Beat via Ballistocardiograph—A hybrid Approach,” 32^{nd} Annual International Conference of the IEEE EMBS Buenos Aires, Argentina, Aug. 31-Sep. 4, 2010, by David Friedrich, Xavier L. Aubert, Hartmut Fuhr and Andreas Brauers, disclosed the use of a single one-dimension (1D) piezoelectric sensor being placed under a mattress in which a person sleeps on in order to measure cardiac micro-pressure being exerted on it through the mattress. The sensor outputs an electronic waveform (BCG signal) which is equivalent to the cardiac micro-pressures. Since this sensor is based on pressure it will not work if it is placed on the surface of the mattress and therefore must be placed under the mattress. However, the mattress thickness of the mattress negatively affects the accuracy of the sensor, and the thicker the mattress the less accurate the sensor will be. Further, this one-dimension sensor can only sense the heart valve closing action that exerts pressure on the mattress, and therefore becomes distorted by various types of noises and interference, as well as body movements on the mattress, which significantly reduce the accuracy of the sensor such that it may not be able to provide real-time heartbeat signals. Also, this single sensor cannot be scaled to remove noise to optimize the cardiac readings.

The article titled: “Monitoring Pulse and Respiration with a Non-Invasive Hydraulic Bed Sensor,” 32^{nd} Annual International Conference of the IEEE EMBS Buenos Aires, Argentina, Aug. 31-Sep. 4, 2010, by David Heise and Marjorie Skubic, disclosed a hydraulic bed sensor to detect cardiac activity. Here a one-dimension (1D) hydraulic transducer sensor is used, which is required to be placed below a mattress so that micro-movements of a person's heart will exert micro-pressure levels on the sensor through the mattress. The hydraulic transducer sensor records these micro-pressure levels. This sensor is based on pressure and therefore must be placed under a mattress. Due to the thickness of a mattress the sensor is not accurate. Further, this one-dimension sensor can only sense the heart valve closing action that exerts pressure on the mattress, and therefore becomes distorted by various types of noises and interference, as well as body movements on the mattress, which significantly reduce the accuracy of the sensor such that it may not be able to provide real-time heartbeat signals. Also, this single sensor cannot be scaled to remove noise to optimize the cardiac readings.

There are also attempts at sleep tracker solutions in the market that use a single Micro Electro-Mechanical System (MEMS) sensor (one dimension (1D)) to sense heart beats and breathing. The MEMS sensor, which is placed under a mattress, can sense heart beats and respiration pulses of a person lying on and sleeping on a mattress. This MEMS device can monitor vital signs without touching a user's body, and therefore is contactless and non-intrusive.

The MEMS sensor is very small and can be placed anywhere under the mattress or on the surface of the mattress, thus rendering similar equipment requiring wires and/or other messy and inconvenient attachments obsolete. The MEMS sensor is better than wearable devices in that it is contactless and therefore does not contact a user's body. Also, the MEMS sensor uses power from a power outlet, and therefore the user does not need to remember to wear it at night or charge any batteries on a regular basis. By using digital signal processing, a MEMS-sensor system can sense heartbeats and respiratory pulses. However, the current 1D-MEMS sensor devices still have many challenging issues in monitoring vital signs in the medical field, including:
1) beds and mattresses generate various types of noises due to different materials and environments which interfere with the sensor signals of the 1D-MEMS sensors;
2) a user's sleeping position changes during a sleep pattern, causing the sensor, which is placed at a stationary location, to become less effective at reading heartbeats or respiratory pulses, and therefore may not be able to sense vital signs consistently or at all. For example, is a user sleeps in the middle of the bed, then the sensor signal may be good, but if the user turns to the right or left during a sleep session, then the amplitude of the sensor will become smaller or even unreadable; and
3) regarding contactless issues, if the 1D-MEMS sensor is placed under a mattress, then the 1D-MEMS sensor must be sensitive enough to sense vital signs through the mattress. However, the sensitivity of the sensor will decrease proportionately with an increase in the thickness of the mattress.

The noise from a mattress and the environment around a 1D-MEMS sensor, the changing positions of a user while lying in on a mattress, and the distance between the sensor and a user's body through the mattress, can cause the 1D-MEMS sensor to become less sensitive and less accurate. If the signal sensed by the 1D-MEMS sensor is not consistently accurate then the product will not be able to provide real time results of vital signs, and will not be able to detect emergency conditions that may occur.

Accordingly, there is a need for a system and method that can sense and record heart-valve activity without requiring pressure.

There is also a need for a system and method that can sense and record heart-valve activity in three dimensions.

There is also a need for a system and method that can sense and record heart-valve activity from a distance and in three dimensions without the requirement of being placed directly under a person.

There is also a need for a system and method that can measure and record blood flow activity as well as heart-valve activity in different dimensions.

There is also a need for a system and method that can combine measurements of heart-valve activity and blood flow activity in different dimensions to obtain a higher accuracy of heartbeats.

There is also a need for a system and method that can “focus” and “virtually steer” sensing to any area and spatial directs (X, Y and Z dimensions) to receive optimal signals at all times.

### SUMMARY OF THE INVENTIVE CONCEPT

The present general inventive concept provides a system and method for sensing human vital signs using an array of 3D-MEMS sensors for remote health monitoring, and more particularly, but not exclusively, this inventive concept provides a system and method for sensing humans' and animals' vital signs using an array of 3D-MEMS sensors for remote health monitoring wherein optimum weights can be calculated for each sensor output to obtain the optimum signal of heartbeats and respiration pulses.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a system to sense human and animal vital signs using an array of three-dimensional sensor units for remote health monitoring, comprising: one or more three-dimensional sensor units each configured to sense heartbeats and respiratory pulses along x, y and z axes from any location on a surface of a mattress or other medium in which a human or animal is on, and each to output three analog signals representing the three dimensions being sensed; three analog-to-digital converters (ADC) corresponding to each of the one or more three-dimensional sensor units to convert the three output analog signals to three digital signals X, Y, Z; a micro-controller unit (MCU) configured to perform digital signal processing (DSP) on the digital signals for each ADC and to output the digitized signals; and a finite impulse response (FIR) low-pass filter (LPF) to receive and low-pass filter each of the output digitized signals.

In an exemplary embodiment, the one or more three-dimensional sensor units includes three three-dimensional sensor units.

In another exemplary embodiment, the three-dimensional sensor units are 3D-Micro Electro-Mechanical System (MEMS) sensor units.

In another exemplary embodiment, the low-pass filtered digitized signals are each assigned variable weights W and then added together by the following formula:

$$Sum\_xyz = Wx 1 * X 1 + Wy 1 * Y 1 + Wz 1 * Z 1 + Wx 2 * X 2 + Wy 2 * Y 2 + Wz 2 * Z 2 + Wx 3 * X 3 + Wy 3 * Y 3 + Wz 3 * Z 3 ,$$

wherein X1, Y1, Z1, X2, Y2, Z2, X3, Y3 and Z3 are the low-pass filtered digitized signals of three three-dimensional sensor units and Wx1, Wy1, Wz1, Wx2, Wy2, Wz2, Wx3, Wy3 and Wz3 are weights added to each corresponding one of the low-pass filtered digitized signals.

In still another exemplary embodiment, the value of the weights Wx1, Wy1, Wz1, Wx2, Wy2, Wz2, Wx3, Wy3 and Wz3 are each assigned to be equal to 1.

In yet another exemplary embodiment, the value of each of the weights Wx1, Wy1, Wz1, Wx2, Wy2, Wz2, Wx3, Wy3 and Wz3 is determined by inserting the system inside a mattress, wherein the characteristics of the mattress is known, and then the optimum value of each weight is determined based on test results collected from a plurality of users on the mattress.

In still another exemplary embodiment, the value of each of the weights Wx1, Wy1, Wz1, Wx2, Wy2, Wz2, Wx3, Wy3 and Wz3 is determined by calculating the signal-to-noise ratio (SNR) for each three-dimensional sensor unit signal as follows: capture and digitize the mattress signals while empty to obtain pure noise signals; calculate signal-to-noise ratio (SNR) for each of the signals where the signals are obtained while a user is on the mattress; find the maximum SNR value (SNR_max) and apply the weight of this signal as equal to 1; and divide each SNR of all the other signals which are not the maximum SNR value to the SNR_max value to obtain the weight of each other signal.

In yet another exemplary embodiment, the MCU is configured to include the nine ADCs embedded therein such that the MCU receives the three analog signals from each of the three three-dimensional sensor units.

In yet another exemplary embodiment, the value of the weights Wx1, Wy1, Wz1, Wx2, Wy2, Wz2, Wx3, Wy3 and Wz3 are each assigned to be equal to 1.

In still another exemplary embodiment, the value of each of the weights Wx1, Wy1, Wz1, Wx2, Wy2, Wz2, Wx3, Wy3 and Wz3 is determined by inserting the system inside a mattress, wherein the characteristics of the mattress is known, and then the optimum value of each weight is determined based on test results collected from a plurality of users on the mattress.

In yet another exemplary embodiment, the value of each of the weights Wx1, Wy1, Wz1, Wx2, Wy2, Wz2, Wx3, Wy3 and Wz3 is determined by calculating the signal-to-noise ratio (SNR) for each three-dimensional sensor unit signal as follows: capture and digitize the mattress signals while empty to obtain pure noise signals; calculate signal-to-noise ratio (SNR) for each of the signals where the signals are obtained while a user is on the mattress; and find the maximum SNR value (SNR_max) and apply the weight of this signal as equal to 1; divide each SNR of all the other signals which are not the maximum SNR value to the SNR_max value to obtain the weight of each other signal.

In still another exemplary embodiment, the system may further include a 3:1 digital multiplexor disposed between each of the ADCs and the MCU configured to receive and digitize and multiplex each of the nine analog signals output by the nine ADCs such that the MCU selects an output of the multiplexor to be input thereto.

In yet another exemplary embodiment, the value of each of the weights Wx1, Wy1, Wz1, Wx2, Wy2, Wz2, Wx3, Wy3 and Wz3 is determined by inserting the system inside a mattress, wherein the characteristics of the mattress is known, and then the optimum value of each weight is determined based on test results collected from a plurality of users on the mattress.

In still another exemplary embodiment, the value of each of the weights Wx1, Wy1, Wz1, Wx2, Wy2, Wz2, Wx3, Wy3 and Wz3 is determined by calculating the signal-to-noise ratio (SNR) for each three-dimensional sensor unit signal as follows: capture and digitize the mattress signals while empty to obtain pure noise signals; calculate signal-to-noise ratio (SNR) for each of the signals where the signals are obtained while a user is on the mattress; and find the maximum SNR value (SNR_max) and apply the weight of this signal as equal to 1; divide each SNR of all the other signals which are not the maximum SNR value to the SNR_max value to obtain the weight of each other signal.

In still another exemplary embodiment, the low-pass filtered digitized signals are each assigned variable weights W and then added together by the following formula: Sum_xyz=Wx1*X1+Wy1*Y1+Wz1*Z1, wherein X1, Y1, Z1 are the low-pass filtered digitized signals of a three three-dimensional sensor unit and Wx1, Wy1, Wz1 are the weights added to each corresponding one of the low-pass filtered digitized signals.

In still another exemplary embodiment, the low-pass filtered digitized signals are each assigned variable weights W and then added together by the following formula:

$$Sum\_xyz = Wx 1 * X 1 + Wy 1 * Y 1 + Wz 1 * Z 1 + Wx 2 * X 2 + Wy 2 * Y 2 + Wz 2 * Z 2 ,$$

wherein X1, Y1, Z1, X2, Y2, Z2 are the low-pass filtered digitized signals of the two three-dimensional sensor units and Wx1, Wy1, Wz1, Wx2, Wy2 are the weights added to each corresponding one of the low-pass filtered digitized signals.

The foregoing and/or other features and utilities of the present general inventive concept may also be achieved by providing a method of sensing human vital signs in three-dimensions for remote health monitoring, the method comprising: sensing heartbeats and respiratory pulse signals of a user on a mattress along x, y and z axes using an array of three-dimensional sensor units and generating analog signals for each of the three-dimensional sensed signals; converting each of the generated analog signals to digital signals; performing digital signal processing on each of the digital signals; and low-pass filtering each of the digital signal processed signals.

### BRIEF DESCRIPTION OF THE DRAWINGS

These and/or other features and utilities of the present inventive concept will become apparent and more readily appreciated from the following description of the embodiments, taken in conjunction with the accompanying drawings of which:

FIG. 1 illustrates a general illustration of a remote system and method to sense human vital signs using an array of 3D-Micro Electro-Mechanical System (MEMS) sensor units, according to an exemplary embodiment of the present inventive concept;

FIG. 2 illustrates a sensor board including an array of 3D-sensors used to sense human vital signs, according to an exemplary embodiment of the present inventive concept;

FIG. 2A illustrates a single 3D-MEMS sensor unit used with the sensor board illustrated in FIG. 2, according to an exemplary embodiment of the present inventive concept;

FIG. 3 illustrates a system to sense human vital signs using an array of 3D-MEMS sensors, according to an exemplary embodiment of the present inventive concept;

FIG. 4 illustrates a system to sense human vital signs using an array of 3D-MEMS sensors, according to another exemplary embodiment of the present inventive concept;

FIG. 5 illustrates a system to sense human vital signs using an array of 3D-MEMS sensors, according to still another exemplary embodiment of the present inventive concept;

FIG. 6 illustrates a finite impulse response (FIR) low-pass filter (LPF) implemented used to filter digitally converted three-dimensional output signals, according to an exemplary embodiment of the present inventive concept;

FIG. 6A illustrates examples of analog-to-digitally converted X, Y and Z signals before being input to the FIR LPF and after being output from the FIR LPF of FIG. 6;

FIG. 7 illustrates a Matlab plot of a summation of output signals of a system to sense human vital signs, according to an exemplary embodiment of the present inventive concept; and

FIG. 8 illustrates a Matlab plot of a summation of output signals of a system to sense human vital signs, according to another exemplary embodiment of the present inventive concept.

The drawings illustrate a few example embodiments of the present inventive concept, and are not to be considered limiting in its scope, as the overall inventive concept may admit to other equally effective embodiments. The elements and features shown in the drawings are to scale and attempt to clearly illustrate the principles of exemplary embodiments of the present inventive concept. In the drawings, reference numerals designate like or corresponding, but not necessarily identical, elements throughout the several views.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

It will be understood that although the terms “first” and “second” are used herein to describe various elements, these elements should not be limited by these terms. These terms are only used to distinguish one element from another element. Thus, a first element could be termed a second element, and similarly, a second element may be termed a first element without departing from the teachings of this disclosure.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part “includes” or “comprises” an element, unless there is a particular description contrary thereto, the part can further include other elements, not excluding the other elements. In the following description, terms such as “unit” and “module” indicate a unit to process at least one function or operation, wherein the unit and the block may be embodied as hardware or software or embodied by combining hardware and software.

Hereinafter, one or more exemplary embodiments of the present general inventive concept will be described in detail with reference to accompanying drawings.

Example embodiments of the present general inventive concept are directed to a system and method for sensing human vital signs using an array of 3D-MEMS sensors for remote health monitoring. The present general inventive concept is also directed to a system and method for sensing humans' and animals' vital signs using an array of 3D-MEMS sensors for remote health monitoring wherein optimum weights can be calculated for each sensor output to obtain the optimum signal of heartbeats and respiration pulses.

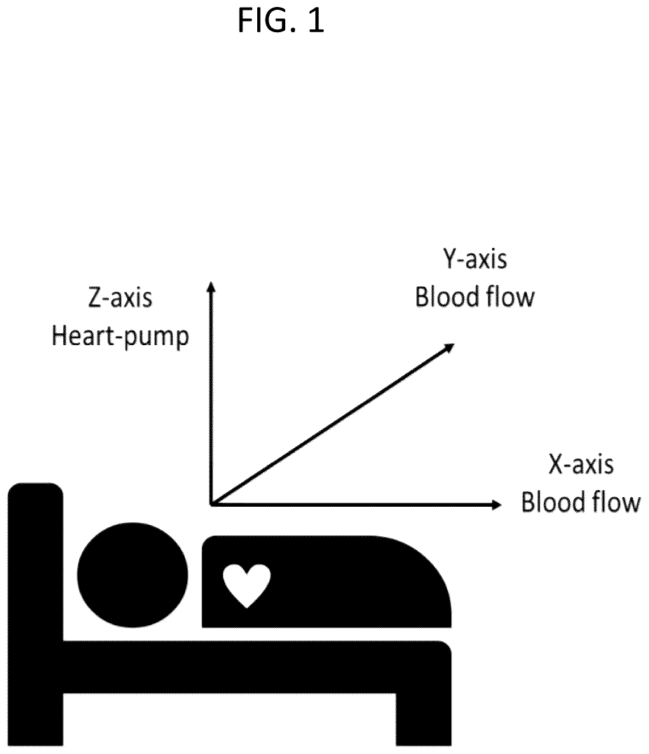

FIG. 1 illustrates a general illustration of a remote system and method to sense human vital signs using an array of 3D-Micro Electro-Mechanical System (MEMS) sensor units for remote health monitoring, according to an exemplary embodiment of the present inventive concept. The remote vital sign sensing system illustrated in FIG. 1 can use an array of 3D-MEMS sensor units each configured for sensing along three axes, including the X, Y and Z axes. According to an exemplary embodiment, the multiple 3D-MEMS sensor units can be placed at different locations, including under a mattress, on the surface of a mattress, or a combination of both under and on the surface of a mattress. The remote vital sign sensing system and method illustrated in FIG. 1 will be described in more detail below with reference to various exemplary embodiments.

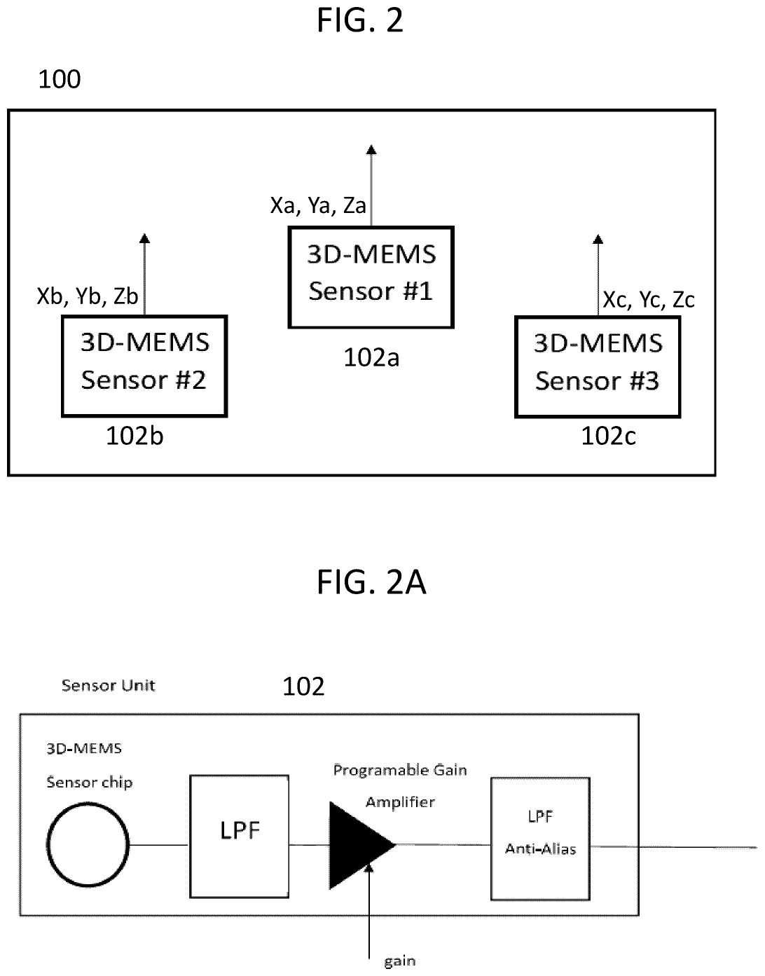

FIG. 2 illustrates a sensor board 100 according to an exemplary embodiment of the present inventive concept. The sensor board 100 is usable together with remote vital sign sensing systems according to various embodiments of the present inventive concept, as will be described in more detail below.

The sensor board 100 illustrated in FIG. 2 can include an array of 3D-MEMS sensor units, which may be placed on the sensor board 100. The sensor board 100 can then be placed under or on the top of a mattress to sense a human's vital signs, including, for example a human's heartbeats and respiratory pulses. In the exemplary embodiment of FIG. 2 the sensor board 100 is illustrated to include an array of three three-dimensional sensor units, a first 3D-MEMS sensor unit 102a which can be placed in a middle area of the sensor board 100, a second 3D-MEMS sensor unit 102b which can be placed on a left side of the sensor board 100, and a third 3D-MEMS sensor unit 102c which can be placed on a right side of the sensor board 100. However, the sensor board 100 can alternatively include two three-dimensional sensors or more than three three-dimensional sensors without departing from the spirit and scope of the present inventive concept.

The sensor board 100 can be placed under a mattress such that the three 3D-MEMS sensor units 102a, 102b and 102c can sense heart beats and respiratory pulses from the left side, middle and right side of a user while the user is sleeping on the mattress. Output signals of the three 3D-MEMS sensor units 102a, 102b and 102c can include Xa, Ya, Za, Xb, Yb, Zb, Xc, Yc and Zc signal outputs. These 3D-MEMS sensor units 102a, 102b and 102c and their output signals Xa, Ya, Za, Xb, Yb, Zb, Xc, Yc and Zc can be combined by different remote vital sign sensing systems and methods according to various exemplary embodiments of the present inventive concept as described below, in order to find the optimum signal of the heartbeats and respiratory pulses. Each of the exemplary embodiments of remote human vital sign sensing systems and methods, as described in more detail below, are configured to find and calculate optimum weights for each sensor output. These optimum weights can be fixed weights or adaptive weights, which can change automatically based on conditions and time.

By combining an array of 3D-MEMS sensor units 102a, 102b and 102c with adaptive weights, various embodiments of the present inventive concept can implement a system that can “focus” and “virtually steer” the sensing capabilities to any area and in any spatial directions (X, Y and Z) so that the exemplary remote human vital sign sensing systems according to the various exemplary embodiments can receive optimum signals at all times. For example, if a user is sleeping on the right side of a mattress, then the remote human vital sign sensing systems according to the various exemplary embodiments can steer the sensing operations to the right side by adapting the weights given to the sensor units 102a, 102b and 102c. Then if a user moves to the left side of the mattress, the remote human vital sign sensing systems can steer the sensing operations to the left side by again adapting the weights given to the sensor units 102a, 102b and 102c. Accordingly, the sensor board 100 illustrated in FIG. 2 can provide an ultra-sensitive mattress sensing system that can measure vital signs accurately and reliably in any conditions and environments.

FIG. 2A illustrates an exemplary embodiment of one of the 3D-MEMS sensor units 102. The 3D-MEMS sensor unit 102 can include a sensor chip having three outputs: X, Y and Z. These three outputs can be analog outputs configured to be the heartbeat signal sensed by the sensor in three-dimensions. Each of the three analog outputs can be input through a low pass filter (LPF) independently to filter out any noise above the frequency of a heartbeat signal. The output from the LPF can then be input through a programmable amplifier (PA) or an automatic gain control (AGC) amplifier. A gain from the PA or AGC can be controlled by a Micro-Control Unit (MCU) according to a specific configuration thereof, as will be described in more detail below. An output of the PA or AGC will be input to an Anti-Alias LPF. In this exemplary embodiment, an output of the Anti-Alias LPF is the output of the 3D-MEMS sensor unit 102. Since there are three sensing signals originating at each 3D-MEMS sensor unit 102, there are three outputs X, Y and Z from each sensor unit 102, which are analog outputs.

The 3D-MEMS sensor unit 102 according to the exemplary embodiment illustrated in FIG. 2 has the capability to sense recoil and vibration in three coordinate axes, independently. For example, the Z-axis senses the heart-pumping action (i.e., the vibration direction from the heart down to the mattress), and when a user is lying on the mattress such that his/her head-to-toe line is aligned with the top-to-bottom line of the mattress the X-axis senses blood flow and volume in the user's arteries through the user's heart (i.e., the recoil action with the heart valves opening and closing). Further, when the user is lying diagonally across the mattress, then a combination of X-axis and Y-axis sensing functions can measure the blood flow and volume of blood in the user's arteries through the heart.

The output signals for the heart-pumping action (Z-axis sensor) and blood flow and volume (X-axis and Y-axis sensors) can be synchronized. For example, peaks of heart-pumping action are naturally aligned with peaks of blood flow and volume, and as such, the system according to the exemplary embodiments of the present inventive concept can combine these 3D signals to obtain a much higher accuracy detection of heartbeats. It is to be noted that although the exemplary embodiment illustrated in FIG. 2 discloses using 3D-MEMS sensor units 102a, 102b and 102c mounted on a sensor board 100, alternative equivalent 3D sensor units, which can perform the intended purposes as described herein, can be used, such as, for example pressure sensors, piezoelectric sensors, optical fiber sensors, etc., without departing from the spirit and scope of the present inventive concept.

Still referring to FIG. 2, according to this exemplary embodiment, an array of three 3D-MEMS sensor units 102a, 102b and 102c is mounted on the sensor board 100. Although three 3D-MEMS sensor units 102a, 102b and 102c are used here, the number of sensor units can alternatively be provided in two, or even in a set of more than three 3D sensor units. In other words, the number of 3D sensor units to be used is flexible depending on the sensing area to be covered.

The sensor board 100 can be placed under a mattress or on a surface of the mattress. If the sensor board 100 is placed on the surface of the mattress, an example of a location on the mattress can be under a pillow or under a user's body, such as under a bed sheet disposed over a mattress.

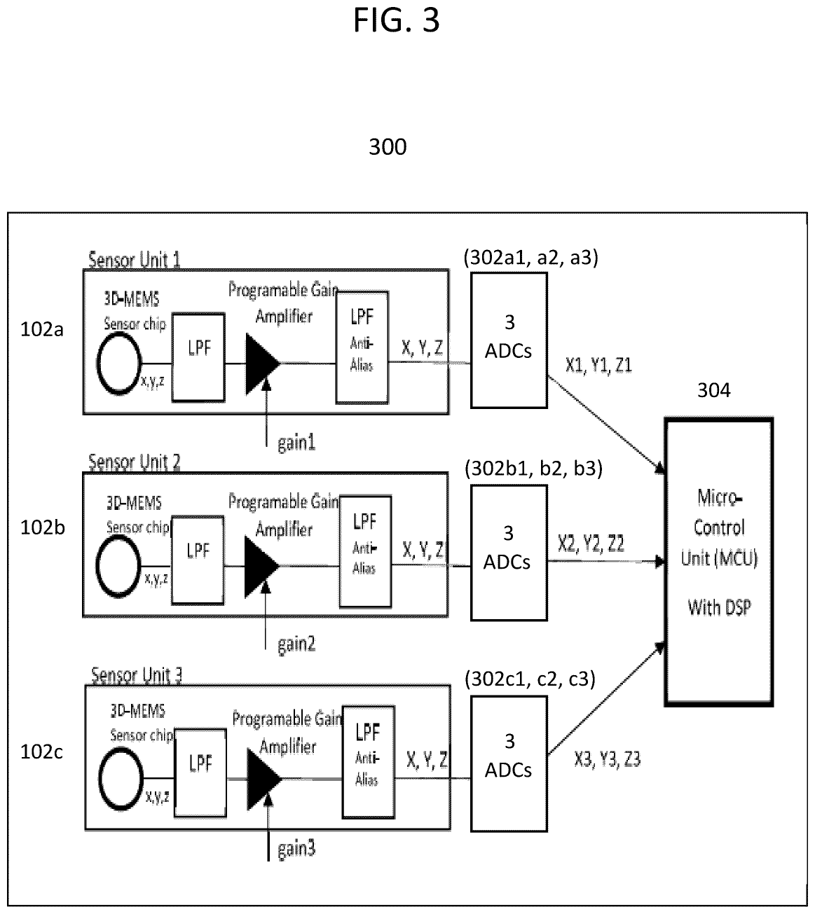

FIG. 3 illustrates a remote human vital sign sensing system 300, according to an exemplary embodiment of the present inventive concept. The remote human vital sign sensing system 300 can include three 3D-MEMS sensor units 102a, 102b and 102c. Each of the three 3D-MEMS sensor units 102a, 102b and 102c are configured to sense heart-pumping action (Z-axis sensor) and blood flow and volume (X-axis and Y-axis sensors), and output three analog signals X, Y and Z. The analog output signals X, Y and Z can then be input to three corresponding analog-to-digital converters (ADC), where the analog signals are converted into digital signals. More specifically, each sensor unit 102a, 102b and 102c will output three analog signals X, Y and Z, and each one of three corresponding ADCs will receive a respective one of the analog signals X, Y and Z. Accordingly, for the three sensor units 102a, 102b and 102c there are nine ADCs, where the sensor unit 102a will be aligned with three ADCs: 302a1, 302a2 and 302a3, the sensor unit 102b will be aligned with three ADCs: 302b1, 302b2 and 302b3 and the sensor unit 102c will be aligned with three ADCs: 302c1, 302c2 and 302c3.

It is to be noted that the number of digital conversion bits output from the ADCs can be tailored based on testing results, as well as on the specifications and sensitivity in which each designer wants to achieve. The nine ADCs will each provide an output, resulting in nine digital signal outputs X1, Y1, Z1, X2, Y2, Z2, X3, Y3 and Z3. These nine digital outputs will be input to a Micro-Controller Unit (MCU) 304 that supports digital signal processing (DSP) calculations.

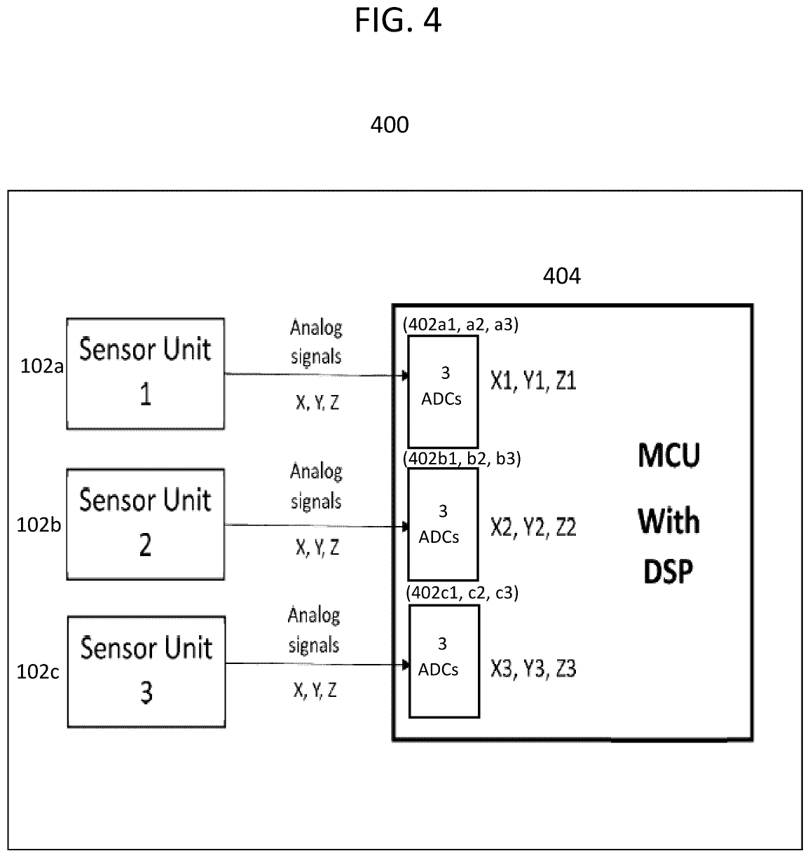

FIG. 4 illustrates an alternative configuration of the remote human vital sign sensing system illustrated in FIG. 3, according to another exemplary embodiment of the present inventive concept. The remote human vital sign sensing system 400 according to the embodiment of FIG. 4 provides a configuration in the case where an MCU does not have a sufficient number of inputs for 9 digital signals X1, Y1, Z1, X2, Y2, Z2, X3, Y3 and Z3, where each digital output signal may comprise, for example, 10 bits of data received from a 10-bit ADC, such that 90 bits of data would be output from the ADCs and be required to be input to the MCU. Accordingly, in this exemplary embodiment an MCU 404 can be configured to include 9 ADCs embedded therein, such that three analog output signals (X, Y, Z) from sensor unit 102a, three analog output signals (X, Y, Z) from sensor unit 102b, and three analog output signals (X, Y, Z) from sensor unit 102c can be input to the MCU 404. More specifically, the MCU 404 can include three ADCs: 402a, 402b and 402c, to receive three output signals X, Y and Z from 3D-MEMS sensor unit 102a, three ADCs: 402b1, 402b2 and 402b3 to receive three output signals X, Y and Z from the 3D-MEMS sensor unit 102b, and three ADCs: 402c1, 402c1 and 402c3 to receive three output signals X, Y and Z from the 3D-MEMS sensor unit 102c. Here the nine analog signals output from sensor units 102a, (X, Y, Z), 102b (X, Y, Z) and 102c (X, Y, Z) can be digitized within the MCU 404 by the nine corresponding ADCs (402a1, 402a2, 402a3), (402b1, 402b2, 402b3) and (402c1, 402c2, 402c3).

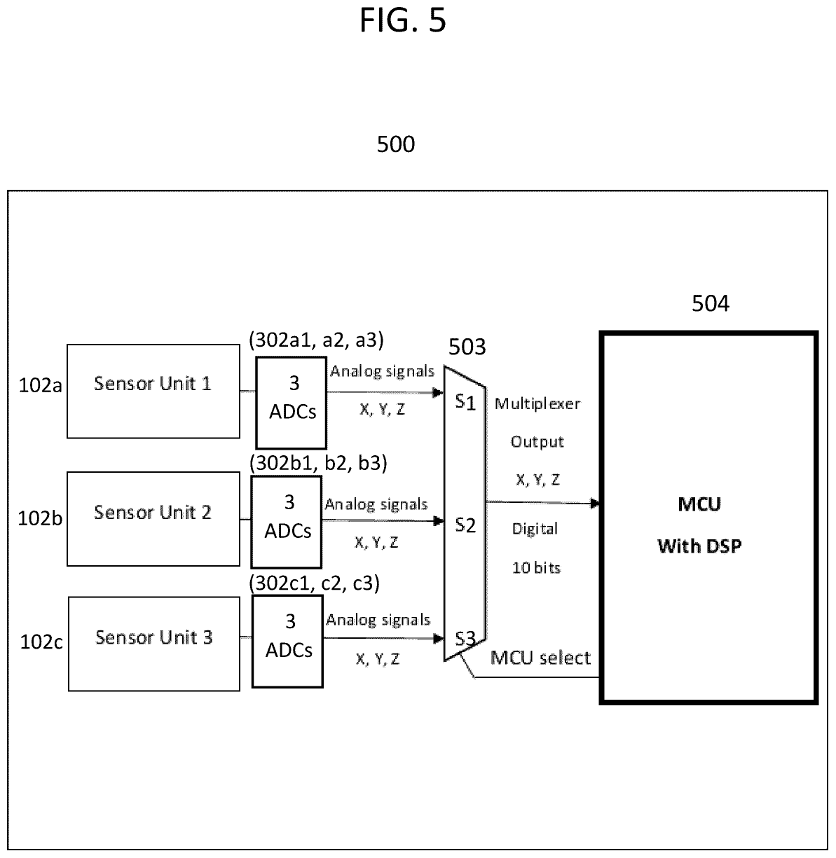

FIG. 5 illustrates another alternative configuration of the remote human vital sign sensing system illustrated in FIG. 3, according to another exemplary embodiment of the present inventive concept. The remote human vital sign sensing system 500 according to the exemplary embodiment of FIG. 5 introduces a digital multiplexer 503 between the ADCs and an MCU 504. The digital multiplexer 503 can be a 3:1 multiplexer. The MCU 504 can use two general-purpose input/output pins to select the output of the multiplexer 503 to be input to the MCU 504. With this configuration the MCU 504 will only require 10 pins for the ADC outputs. In other words, the MCU 504 can select from among one of the inputs S1, S2 or S3 to be input to the MCU 504.

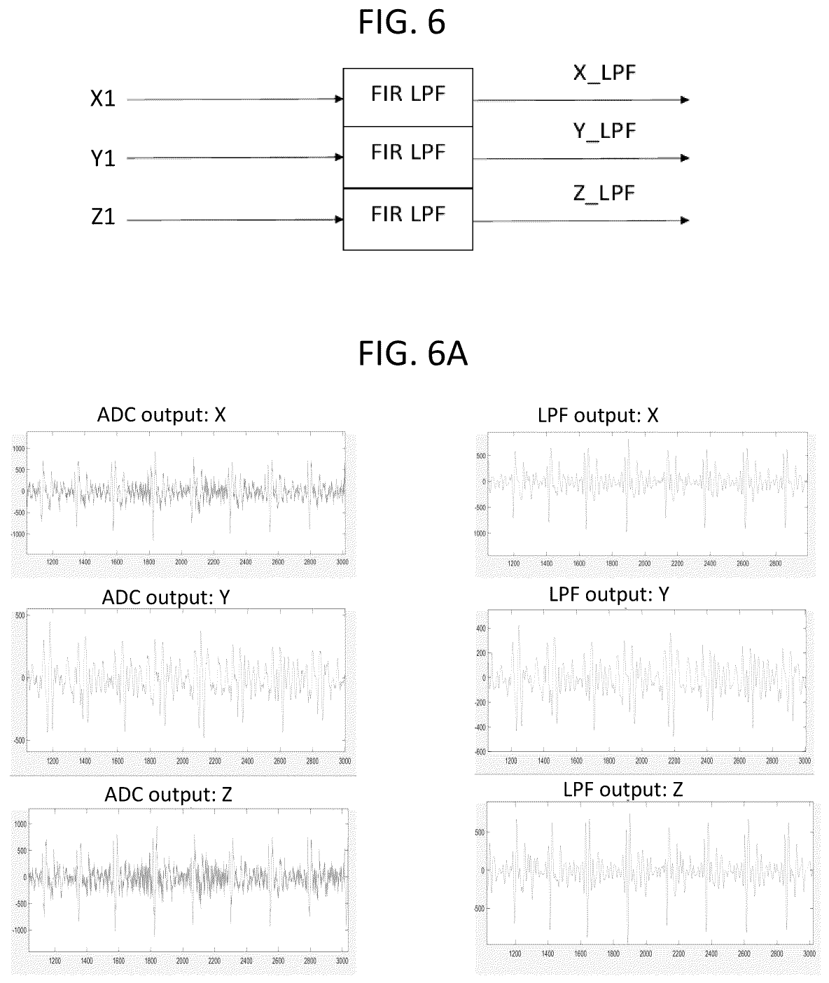

FIG. 6 illustrates a low-pass filter (LPF) 600 implemented by firmware using a finite impulse response (FIR) method. Once the ADC outputs (X1, Y1, Z1), (X2, Y2, Z2) and (X3, Y3, Z3) are received by the MCU 304 or MCU 504, or in the case of the exemplary embodiment of FIG. 4 where the digitizing occurs within the MCU 404, these digitized signals can be further filtered by the low-pass filter (LPF) 600. As is well known, the low-pass filter has a cut-off frequency point of 0.707 or −3 dB (dB=−20 log*V_{OUT/IN}) of the voltage gain allowed to pass. The 3 dB cutoff frequency of the LPF 600 is between 10 Hz to 20 Hz depending on its application and test results.

FIG. 6A illustrates examples of the analog-to-digitally converted X, Y and Z signals before being input to the FIR LPF and after being output from the FIR LPF (X_LPF, Y_LPF, Z_LPF).

The LPF outputs (X1_LPF, Y1_LPF, Z1_LPF) can then be combined to achieve optimum heartbeat waveforms. More specifically, a variable weight W can be assigned to each waveform in the following manner:

$$Sum\_xyz = Wx 1 * X 1 + Wy 1 * Y 1 + Wz 1 * Z 1 + Wx 2 * X 2 + Wy 2 * Y 2 + Wz 2 * Z 2 + Wx 3 * X 3 + Wy 3 * Y 3 + Wz 3 * Z 3.$$

There are several methods to determine the values of these variable weights to combine multiple sensor outputs. A first method is to assign fixed equal weights to all the signals. For example, each weight can be equal to 1. Here the combined signal is as follows:

Sum_xyz=X1+Y1+Z1+X2+Y2+Z2+X3+Y3+Z3.

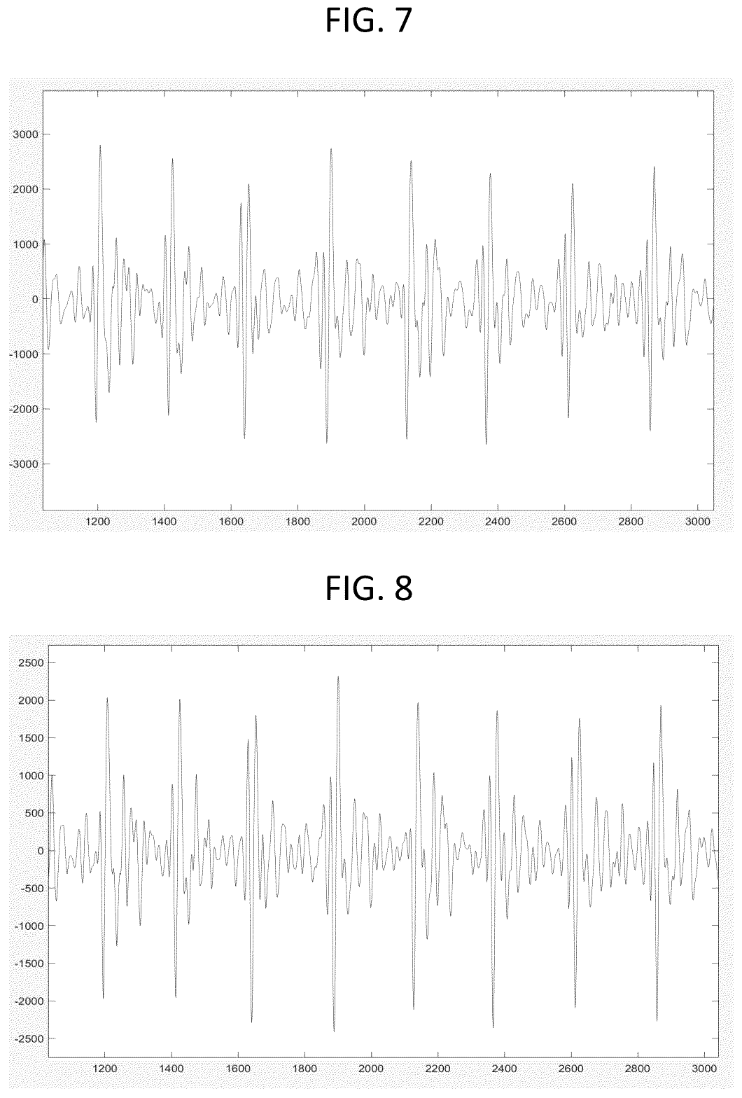

FIG. 7 illustrates a Matlab plot of the summation Sum_xyz of all signals where the weight is equal to 1. Heartbeats can be detected from this waveform.

A second method is an example where the remote human vital sign sensing system according to the exemplary embodiments is embedded inside a mattress, where the characteristics of the mattress and signals are known in advance. Here the optimum value of each weight is searched based on test results collected from many users on the specific mattress. In this case the values of the weights may be different from each other:

$$Sum\_xyz = Wx 1 * X 1 + Wy 1 * Y 1 + Wz 1 * Z 1 + Wx 2 * X 2 + Wy 2 * Y 2 + Wz 2 * Z 2 + Wx 3 * X 3 + Wy 3 * Y 3 + Wz 3 * Z 3$$

where the weight W of each signal may be different from the others.

A third method uses empty-mattress signals to calculate signal-to-noise ratio (SNR) for each sensor signal and then calculates the values of weights based on the SNR results. More specifically, at calibration time or when no user is on the mattress, the MCU can capture and digitize the empty mattress signals using the same paths as heartbeat waveforms. These empty mattress signals are considered as noise, such as: eb_X1, eb_Y1, eb_Z1, . . . , eb_X3, eb_Y3, and eb_Z3.

Then the signal-to-noise ratio (SNR) for each signal is calculated, where the signals are X1, Y1 Z1, X2, Y2, Z2, X3, Y3 and Z3 and the noises are eb_X1, eb_Y1, eb_Z1, . . . , eb_X3, eb_Y3, and eb_Z3. The SNR can be calculated in the time domain or in the frequency domain.

Next the maximum SNR value (SNR_max) is found. The signal that is related to the SNR_max will have a weight equal to 1. The SNR of each sensor signal calculated for the empty mattress is then divided by SNR_max to obtain the weight of that signal. More specifically: if X1 has a maximum SNR, then Wx1=1, and the other weights can be calculated as follows:

Wx1=1;Wy1=SNRy1/SNR_max;Wz1=SNRz1/SNR_max;

Wx2=SNRx2/SNR_max; Wy2=SNRy2/SNR_max; Wz2=SNRz2/SNR_max;

Wx3=SNRx3/SNR_max; Wy3=SNRy3/SNR_max; Wz3=SNRz3/SNR_max.

Therefore, Sum_xyz will be as following:

$$Sum\_xyz = Wx 1 * X 1 + Wy 1 * Y 1 + Wz 1 * Z 1 + Wx 2 * X 2 + Wy 2 * Y 2 + Wz 2 * Z 2 + Wx 3 * X 3 + Wy 3 * Y 3 + Wz 3 * Z 3.$$

FIG. 8 illustrates a Matlab plot of the summation Sum_xyz of all signals using variable weights based on the SNR calculation and the empty mattress signal.

After the combined signal is optimized, the heartbeat pulses can be detected from this optimized signal.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A system to sense human and animal vital signs using an array of three-dimensional sensor units for remote health monitoring, comprising:
one or more three-dimensional sensor units each configured to sense heartbeats and respiratory pulses along x, y and z axes from any location on a surface of a mattress or other medium in which a human or animal is on, and each to output three analog signals representing the three dimensions being sensed;
three analog-to-digital converters (ADC) corresponding to each of the one or more three-dimensional sensor units to convert the three output analog signals to three digital signals X, Y, Z;
a micro-controller unit (MCU) configured to perform digital signal processing (DSP) on the digital signals for each ADC and to output the digitized signals; and
a finite impulse response (FIR) low-pass filter (LPF) to receive and low-pass filter each of the output digitized signals.

2. The system according to claim 1, wherein the one or more three-dimensional sensor units includes three three-dimensional sensor units.

3. The system according to claim 2, wherein the three-dimensional sensor units are 3D-Micro Electro-Mechanical System (MEMS) sensor units.

4. The system according to claim 2, wherein the low-pass filtered digitized signals are each assigned variable weights W and then added together by the following formula:
$$Sum\_xyz = Wx 1 * X 1 + Wy 1 * Y 1 + Wz 1 * Z 1 + Wx 2 * X 2 + Wy 2 * Y 2 + Wz 2 * Z 2 + Wx 3 * X 3 + Wy 3 * Y 3 + Wz 3 * Z 3 ,$$
wherein X1, Y1, Z1, X2, Y2, Z2, X3, Y3 and Z3 are the low-pass filtered digitized signals of three three-dimensional sensor units and Wx1, Wy1, Wz1, Wx2, Wy2, Wz2, Wx3, Wy3 and Wz3 are weights added to each corresponding one of the low-pass filtered digitized signals.

5. The system according to claim 4, wherein the value of the weights Wx1, Wy1, Wz1, Wx2, Wy2, Wz2, Wx3, Wy3 and Wz3 are each assigned to be equal to 1.

6. The system according to claim 4, wherein the value of each of the weights Wx1, Wy1, Wz1, Wx2, Wy2, Wz2, Wx3, Wy3 and Wz3 is determined by inserting the system inside a mattress, wherein the characteristics of the mattress is known, and then the optimum value of each weight is determined based on test results collected from a plurality of users on the mattress.

7. The system according to claim 4, wherein the value of each of the weights Wx1, Wy1, Wz1, Wx2, Wy2, Wz2, Wx3, Wy3 and Wz3 is determined by calculating the signal-to-noise ratio (SNR) for each three-dimensional sensor unit signal as follows:
capture and digitize the mattress signals while empty to obtain pure noise signals;
calculate signal-to-noise ratio (SNR) for each of the signals where the signals are obtained while a user is on the mattress; and
find the maximum SNR value (SNR_max) and apply the weight of this signal as equal to 1; divide each SNR of all the other signals which are not the maximum SNR value to the SNR_max value to obtain the weight of each other signal.

8. The system according to claim 2, wherein the MCU is configured to include the nine ADCs embedded therein such that the MCU receives the three analog signals from each of the three three-dimensional sensor units.

9. The system according to claim 8, wherein the value of the weights Wx1, Wy1, Wz1, Wx2, Wy2, Wz2, Wx3, Wy3 and Wz3 are each assigned to be equal to 1.

10. The system according to claim 8, wherein the value of each of the weights Wx1, Wy1, Wz1, Wx2, Wy2, Wz2, Wx3, Wy3 and Wz3 is determined by inserting the system inside a mattress, wherein the characteristics of the mattress is known, and then the optimum value of each weight is determined based on test results collected from a plurality of users on the mattress.

11. The system according to claim 8, wherein the value of each of the weights Wx1, Wy1, Wz1, Wx2, Wy2, Wz2, Wx3, Wy3 and Wz3 is determined by calculating the signal-to-noise ratio (SNR) for each three-dimensional sensor unit signal as follows:
capture and digitize the mattress signals while empty to obtain pure noise signals;
calculate signal-to-noise ratio (SNR) for each of the signals where the signals are obtained while a user is on the mattress; and
find the maximum SNR value (SNR_max) and apply the weight of this signal as equal to 1; divide each SNR of all the other signals which are not the maximum SNR value to the SNR_max value to obtain the weight of each other signal.

12. The system according to claim 2, further comprising:
a 3:1 digital multiplexor disposed between each of the ADCs and the MCU configured to receive and digitize and multiplex each of the nine analog signals output by the nine ADCs such that the MCU selects an output of the multiplexor to be input thereto.

13. The system according to claim 11, wherein the value of the weights Wx1, Wy1, Wz1, Wx2, Wy2, Wz2, Wx3, Wy3 and Wz3 are each assigned to be equal to 1.

14. The system according to claim 11, wherein the value of each of the weights Wx1, Wy1, Wz1, Wx2, Wy2, Wz2, Wx3, Wy3 and Wz3 is determined by inserting the system inside a mattress, wherein the characteristics of the mattress is known, and then the optimum value of each weight is determined based on test results collected from a plurality of users on the mattress.

15. The system according to claim 11, wherein the value of each of the weights Wx1, Wy1, Wz1, Wx2, Wy2, Wz2, Wx3, Wy3 and Wz3 is determined by calculating the signal-to-noise ratio (SNR) for each three-dimensional sensor unit signal as follows:
capture and digitize the mattress signals while empty to obtain pure noise signals;
calculate signal-to-noise ratio (SNR) for each of the signals where the signals are obtained while a user is on the mattress; and
find the maximum SNR value (SNR_max) and apply the weight of this signal as equal to 1; divide each SNR of all the other signals which are not the maximum SNR value to the SNR_max value to obtain the weight of each other signal.

16. The system according to claim 1, wherein the low-pass filtered digitized signals are each assigned variable weights W and then added together by the following formula:
Sum_xyz=Wx1*X1+Wy1*Y1+Wz1*Z1,
wherein X1, Y1, Z1 are the low-pass filtered digitized signals of a three three-dimensional sensor unit and Wx1, Wy1, Wz1 are the weights added to each corresponding one of the low-pass filtered digitized signals.

17. The system according to claim 1, wherein the one or more three-dimensional sensor units includes two three-dimensional sensor units and the low-pass filtered digitized signals are each assigned variable weights W and then added together by the following formula:
$$Sum\_xyz = Wx 1 * X 1 + Wy 1 * Y 1 + Wz 1 * Z 1 + Wx 2 * X 2 + Wy 2 * Y 2 + Wz 2 * Z 2 ,$$
wherein X1, Y1, Z1, X2, Y2, Z2 are the low-pass filtered digitized signals of the two three-dimensional sensor units and Wx1, Wy1, Wz1, Wx2, Wy2 are the weights added to each corresponding one of the low-pass filtered digitized signals.

18. A method of sensing human vital signs in three-dimensions for remote health monitoring, the method comprising:
sensing heartbeats and respiratory pulse signals of a user on a mattress along x, y and z axes using an array of three-dimensional sensor units and generating analog signals for each of the three-dimensional sensed signals;
converting each of the generated analog signals to digital signals;
performing digital signal processing on each of the digital signals; and
low-pass filtering each of the digital signal processed signals.
